(19) United States
(12) Patent Application Publication
(10) Pub. No.: US 2025/0374893 A1
(43) Pub. Date: Dec. 11, 2025

(54) Pet Collar Reminder Light with Cellphone Application

(71) Applicants: Erin Troy, Walnut Creek, CA (US); John Ross, Walnut Creek, CA (US); Patrick Maher, Walnut Creek, CA (US)

(72) Inventors: Erin Troy, Walnut Creek, CA (US); John Ross, Walnut Creek, CA (US); Patrick Maher, Walnut Creek, CA (US)

(21) Appl. No.: 18/738,913

(22) Filed: Jun. 10, 2024

(51) Int. Cl. A01K 27/00; G08B 21/24; G16H 20/10

(52) U.S. Cl. CPC A01K 27/006; G08B 21/24; G16H 20/10

(57) ABSTRACT

A portable, programable collar LED light assembly and programmable application to create reminders for an event or activity, such as the administration of medication, that requires an action to turn off the reminder.

### FIELD OF THE INVENTION

This invention relates generally to the field of portable LED lights and more specifically to a collar light and application system and method.

### CROSS REFERENCE TO RELATED APPLICATIONS

This application claims the benefit of U.S. Provisional Patent Application No. 63/472,232, filed Jun. 9, 2023, entitled “Pet Collar Reminder Light with Cellphone Application”, the disclosure of which is incorporated by reference herein in its entirety.

### BACKGROUND

Various types of portable LED lights have been designed for many special applications. They are popular because they are small, lightweight and require a relatively small amount of electrical power. Some recent LED lights and devices incorporating LED lights have been designed for use with pet devices, such as an illuminated leash and even an illuminated dog collar. However, there is a deficiency in the prior technology in that none of the existing LED light-incorporating devices can be worn by a pet to illuminate and remind the owner that it is time to provide medications or other types of actions. Additionally, none of the existing lights for pets includes a programmable application that can remotely set different times for notifications to occur and can also have the notifications occur on the cellphone or other smart device in text or audible forms.

Pets sometimes have illnesses or conditions that require medication administration or other actions. Some medications require a single dose, but many medications require multiple administrations. Some medication administration events occur multiple times in one day. These administrations can be difficult to remember, especially if there are multiple medications with different administration regimes. Also, as the schedules of people seem to become increasingly hectic and daily events seem to increase, people can be inconsistent with remembering medication administration times. However, sometimes people forget their pet's requirements if it is not part of the daily routine, as illnesses often are, as well as just being busy and not focusing what they are doing. Likewise, even if a pet is not ill, sometimes it is desirable to have a reminder for an action regarding a pet.

Clients have said to veterinarians, “I wish my dog could remind me!” At least one veterinarian has received confessions that normal cellphone reminder programs work and the alarms go off, but the cellphone users simply choose to ignore them. If the cellphone reminder was used for a pet medication regime, and the cellphone user simply hits the “snooze” button or the “alarm off” button, the purpose for the alarm, and thus the required pet medication administration, may well be forgotten and the administration window of opportunity missed. People need help remembering and what better reminder than the pet itself? What is needed is a system and method for notification of pet-related events, such as medication administration.

### SUMMARY

The current invention relates to systems and methods for a programable LED light assembly, in conjunction with a programmable application (app), to create reminders for an event or activity, such as the administration of medication, that requires an action to turn off the reminder.

Another objective of the invention is to provide an LED light assembly that is programmable.

Yet another objective of the invention is to provide an LED light assembly that provides an application that shows text as a reminder.

Still another objective of the invention is to provide an LED light assembly that provides an application that produces audio sounds as a reminder.

Another objective of the invention is to provide an LED light assembly that provides an application that produces at least one light display as a reminder.

A further objective of the invention is to provide an LED light assembly that is relatively low in weight.

Yet another objective of the invention is to provide an LED light assembly that is weather-resistant.

Another objective of the invention is to provide an LED light assembly that is water-resistant.

Yet another objective of the invention is to provide an LED light assembly and corresponding application for a mobile device that requires the user to disable the reminder on both the LED light assembly and the corresponding application.

Another objective of the invention is to provide an LED light assembly and corresponding application for a mobile device that requires the user to disable the reminder on either the LED light assembly or the corresponding application.

In still another embodiment, the collar light batteries charge wirelessly. In yet still another embodiment, the PCB is flexible, the battery array is flexible, and the housing is flexible. In another embodiment, the solder used in the collar light is flexible. In another embodiment, the PCB, battery array, solder, and housing are flexible, and the battery array charges wirelessly.

The invention preferred embodiment is a pet collar medication reminder light (collar light) with an electronics housing, LED housing, LED lights, a printed circuit board, processor, a top lens, a power source, a connection means for the printed circuit board, a charging means to charge a rechargeable battery and processor communicatively coupled to at least one of server and/or network module via network as well as communicatively coupled to at least one of server and/or mobile device via UWB, BLE, Bluetooth, Wifi, or a combination of UWB and BLE. The device may also include an attachment solution to communicatively couple the device with an external processor.

The LED lights are powered by a battery located within the electronics housing. In the preferred embodiment, the attachment of the light electronics housing holds it to a pet collar so that the LED lights can shine brightly from the collar to alert the owner of the reminder, but the invention can be attached to things other than a pet collar. In the preferred embodiment, no sounds emanate from the collar light, but this feature may be incorporated in alternative embodiments.

In the preferred embodiment, the smart device App also buzzes, chimes, or barks with photos of the pet and words that say “MY MEDICATION IS DUE NOW” as well as other messages. Once the medication is given, the reminder is turned off on the app itself, and the light will have to be physically turned off on the collar light as well. This is to encourage compliance. There are other options for the reminders on the app such as: TIME TO WALK, TIME FOR FEEDING or TIME FOR BATH, and other messages, including custom messages. In another embodiment, multiple messages can be scheduled and set.

In the preferred embodiment, the pet reminder cell phone application (app) is programmable and provides instructions to the collar light via bluetooth technology. In this exemplary embodiment, the mobile device computing application program will allow a reminder to be set at varying times, for example, every 12 hours, 8 hours, 6 hours, 4 hours, and 2 hours, etc. The program allows the schedule to be repeated for a select number of days or longer terms.

Other aspects, objectives and advantages of the present invention will become apparent from the following descriptions, taken in connection with the accompanying drawings, wherein, by way of illustration and example, an embodiment of the present invention is disclosed. Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1 is the top perspective of the invention that shows the LED light and power switch.

FIG. 2 is the back perspective of the invention that shows the LED light and power switch.

FIG. 3 is the front perspective of the invention that shows the retainment clasp.

FIG. 4 is a first side perspective of the invention.

FIG. 5 is a second side perspective of the invention that shows the charging port.

FIG. 6 is the bottom perspective of the invention that shows the housing bottom attachment.

FIG. 7 is an exploded view of the invention.

FIG. 8 is a block diagram illustrating the system architecture according to one embodiment.

### DETAILED DESCRIPTION AND INSTRUCTIONS

The present invention provides a novel and efficient system and method for a programable reminder light system, including reminders to administer pets their medications at predetermined times and intervals. In the preferred embodiment, the pet wearable device attaches directly to the pet's collar. The entire device is lightweight and can fit on dorsal or ventral (or even directed to a right or left lateral side) on the pet's neck on a wide variety of pet collars. The light emitted from the invention helps the pet owner to be reminded of an event, which could be a one-time event or, more likely, a reoccurring event. One such reoccurring event is the administration of a medication, and therein that medicine should now be given to the pet. In another embodiment, the app will also remind the pet owner (or pet sitter), via text and sound emanating from the phone, that it is time to administer medication.

The device is configured to include a processor and at least a sensor which the processor is configured to transmit at least one reminder signal to the pet owner. In accordance with a preferred embodiment of the invention, there is disclosed a pet collar reminder light comprising of: a light-emitting diode (“LED”) housing, an electronics housing and attached LED housing retaining frame, a printed circuit board (“PCB”), a processor, a lens, a plurality of LED lights, a rechargeable battery, an attachment solution, an on/off switch, and an LED light retainer solution. In some embodiments, the on/off switch serves multiple functions besides turning the reminder on and off. The LED retainer solution is designed to retain the LEDs within the LED housing and direct the LEDs to face away from the pet medial axis (facing upward, downward or outward from the pet) so that the user can clearly see the LED reminder lights. In the preferred embodiment, the pet collar reminder light contains a PCB that allows the pet wearable device to interface with a mobile device-based application. In the preferred embodiment, the pet wearable device PCB allows the pet wearable device to connect to a user mobile device, preferably through Bluetooth chip/Bluetooth enabled. However, the pet wearable device may also connect to a user mobile device using any number of protocols, including UWB, BLE, Bluetooth, Wifi, or a combination of UWB and BLE. Android and iOS cellphones, or other smart devices, can be utilized with the application. In the preferred embodiment, this connection protocol uses a security protocol, such as encryption, between the pet wearable device and the user mobile device.

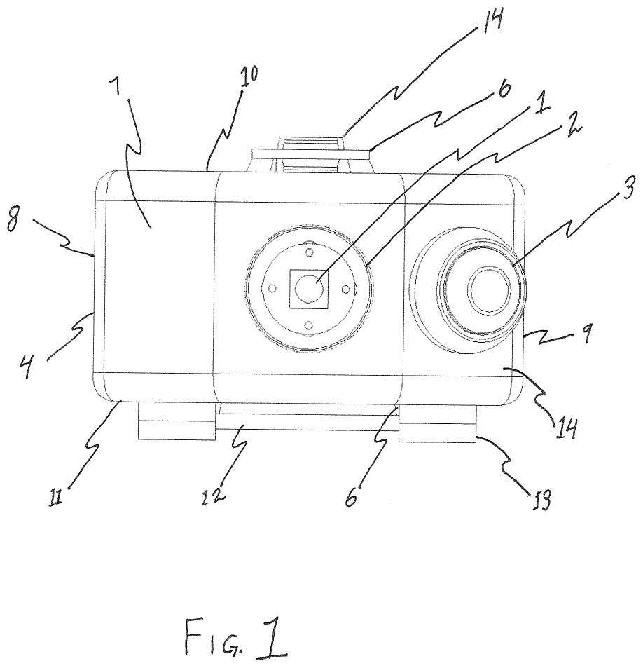

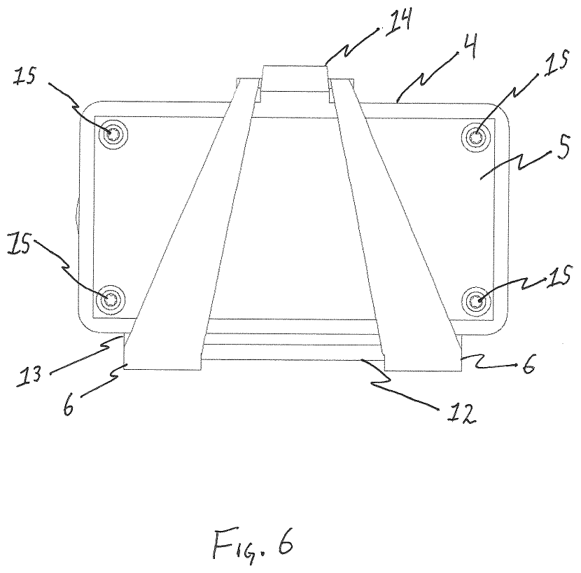

Now looking at FIG. 1, a top view of the preferred embodiment displays the invention from the user vantage point. Specifically, looking down upon the pet wearable device, the LED (1) is located within the LED lens (2). In this embodiment, the on/off switch (3) is a button that provides a means to turn off, reset or deselect a notification. The LED (1), LED lens (2), and on/off switch (3) are located on the device housing (4). The housing has a top (7), a first side (8), a second side (9), a front (10), and a back (11). The housing also has a housing bottom portion (5) that not visible in this figure. (see FIG. 6). In this embodiment, the on/off switch (3) button is located on a housing facet (14) between the top (7) and the second side (9). The attachment solution (6) is used in this embodiment to attach the pet wearable device to a pet collar (not shown). The attachment solution (6) is connected to the housing (4) at opposing sides of the housing, being the front (10) and the back (11). Specifically, the attachment solution (6) is connected to the back (11) by the attachment solution rod (12) being retained by the mounting anchor (13), which is attached to the housing. (4) The other end of the attachment solution (6) is connected to the front (10) at the retaining clasp. (14)

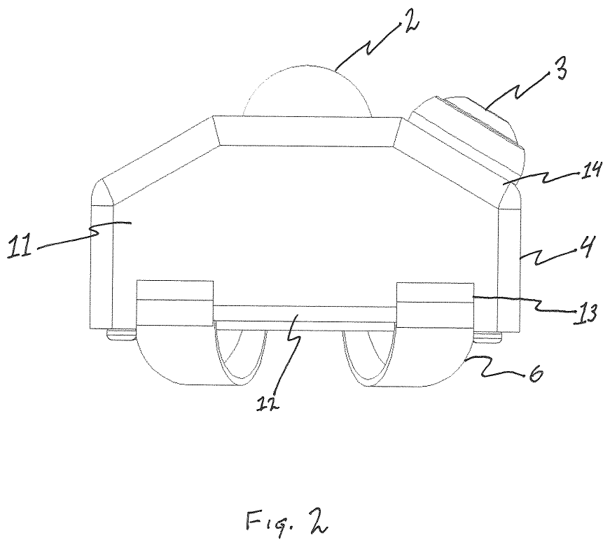

Now referring to FIG. 2, from the housing back (11), the LED lens (2) and on/off switch (3) are seen in this embodiment on the top (7) and housing facet (14), respectively. The attachment solution rod (12) of the attachment solution (6) is retained by the mounting anchor. (13)

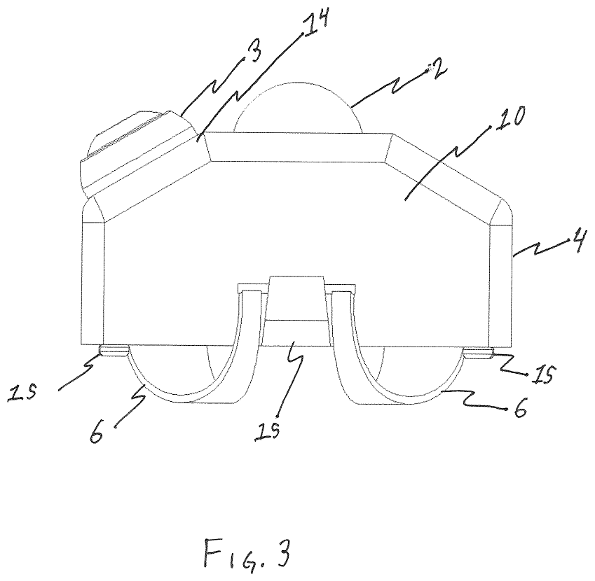

Now referring to FIG. 3, which displays the front (10) of the housing (4), as can be assumed in this embodiment, similarly displays many of the features that are seen from the back (11) of the housing (4) in FIG. 2, such as the LED lens (2), on/off switch (3), housing facet (14), and attachment solution (6). However, from this front (10) view, the retainment clasp (15) for the attachment solution (6) is seen. In this embodiment, the retainment clasp (15) is configured to semi-permanently retain the attachment solution (6) on the pet collar when in use, but also may be disengaged to detach the pet wearable device from the pet collar. In this embodiment, screws (16) that hold the housing bottom (5) can be seen.

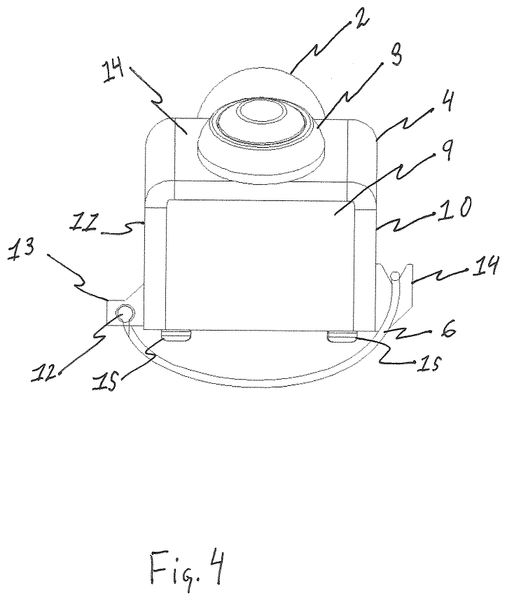

FIG. 4 displays the second side (9) of the housing (4) and the housing facet (14) so that the on/off switch (3) and LED lens (2) are clearly visible, as are the screws (15) that secure the housing bottom (5) to the housing (4). The attachment solution (6) can be seen spanning the front (10) and the back (11) of the pet wearable device housing (4). The attachment solution rod (12) is retained by the mounting anchor (13) on the back (11) and the retainment clasp (14) on the front (10).

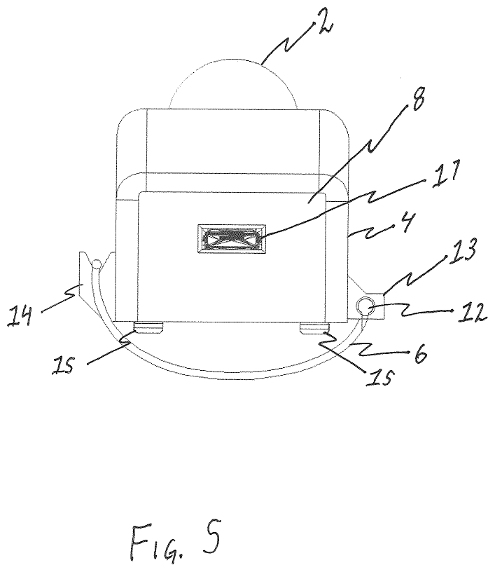

FIG. 5 shows the first side (8) of the housing (4) and, in a similar fashion as the comparison of FIG. 2 and FIG. 3, displays many of the same features as FIG. 4, including the LED lens (2), screws (15), attachment solution (6), attachment solution rod (12), mounting anchor (13), and retainment clasp (14). However, in this embodiment, the charging port (17) is located on the first side (8).

Finally, looking at FIG. 6, the bottom (5) of the housing (4) is viewed. Looking now at the bottom of the pet wearable device, the attachment solution (6) span of the front (10) and the back (11) across the bottom (5) is displayed, as are the two attachment points of the attachment solution (6) at the retainment clasp (14) and the mounting anchor. (13) The attachment solution rod (12) is shown being retained by the mounting anchor. (13)

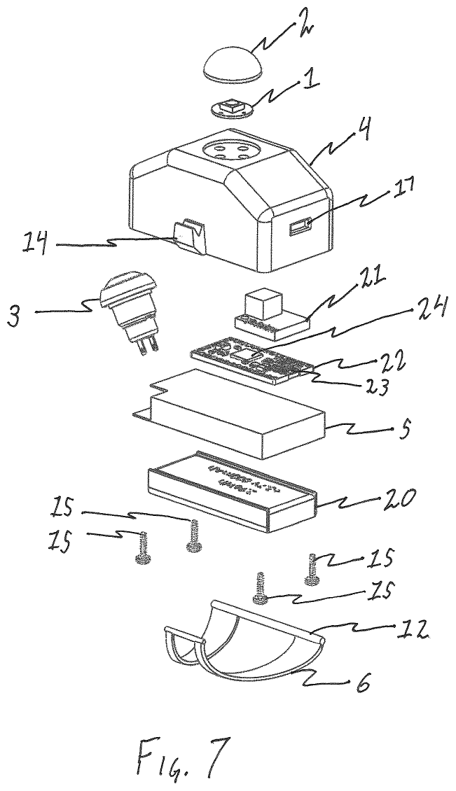

FIG. 7 displays an exploded view of the pet wearable device. In this view, many features of the pet wearable device described above are seen, such as the housing (4), LED (1), LED lens (2), charging port (17), retainment clasp (14), on/off switch (3), screws (15), bottom (5), attachment solution rod (12), and attachment solution. (6) From this view, the power source battery (20) is shown. The charging port connector (21) configured to correspond with the charging port (17) for charging the battery (20) with the use of a charging cord (not shown). The circuit board and microcontroller (22), in this embodiment with a memory (23) and processor (24) is also displayed.

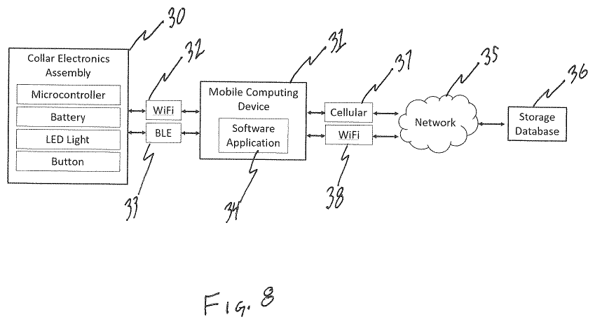

Now turning to FIG. 8, the pet wearable device notification system and method is depicted, according to an exemplary embodiment. The system and method includes the pet wearable device, here termed the collar electronics assembly (30), is shown to include the microcontroller (22), battery (20), LED (1) light, and on/off switch (3) button. The system and method includes a user mobile computing device (31) communicatively coupled with the pet wearable device (30). The user mobile computing device (31) has a software application (34) installed that is designed to function as described herein as well as is configured to communicatively connect and facilitate data usage and exchange for the programming and notifications functions of the system and method as also described herein. In this preferred embodiment, the user mobile computing device (31) and the pet wearable device (3)) are communicatively coupled by WiFi (32) and BLE (32), but other communication solutions can be employed in other embodiments. In this preferred embodiment, the system and method further includes a server coupled with a storage database (36) and a communicative network. (35) In this exemplary embodiment, the network (35) is communicatively connected to the user mobile computing device (31) by cellular (37) and WiFi (38) network protocols designed to exchange data. However, this system is not limited to pets as described herein and may be used with children, elderly individuals, or any other applicable body or item that may require monitoring or notifications.

In the preferred embodiment, the pet wearable device can be affixed to top or bottom of the collar via the attachment solution. The entire invention of the pet wearable device is designed to be lightweight and easily fit on and attach to a wide variety of pet collars. In the preferred embodiment, the dimensions of the preferred embodiment are approximately 2 inches long, 1 inch high, and 1 inch wide, but other embodiments can have different dimensions.

Once the pet wearable device LED (1) light illuminates, it will stay on until physically turned off or the power supply is depleted. This will ensure that even though the pet owner receives the alert via the app and turns it off, the light will remain on to again remind the owner that the event reminder is activated, such as pet medication is required. Functionally, this equates to a 2-factor compliance using both the app and the button on the device to turn off the notification.

The pet wearable device has a reminder on and off switch (3) located on the main housing. (4) In the preferred embodiment, the battery and reminder on/off switch (3) are located on the main housing (4), and the on/off switch (3) can turn on or off the LED (1) lights. In the preferred embodiment, an integrated button on/off switch (3) is located on the housing facet (14), however, the on/off switch (3) can be located anywhere on the pet wearable device in other embodiments. In the preferred embodiment, the LED (1) light uses a rechargeable lithium-ion battery. In the preferred embodiment, the battery is recharged using a USB-C Charging port. The battery may consist of a single battery or a plurality of batteries, including an array of batteries. However, various batteries can be used, and charging can also be wireless.

In the preferred embodiment, the mobile device application allows for the following functions: (1) set the day, time of day and frequency of times that a pet must be given medication; (2) when a reminder notification is due (such as administration of medication), it will convey the message, such as showing a page, that states the preprogramed message (such as medication is due) and will also emit an audible sound on the mobile device application; (3) the pet owner may customize the mobile device application program with text and photos; and, (5) the app can be used for other functions that require a time notification, e.g., infant feedings, senior medication times, and automated pharmacy orders. In an alternative embodiment, the pet's entire medical history can be contained in the application.

The method and system may comprise at least one mobile computer device (31) operable to display template screens in and on a software application (34) that permit users to configure the types of event notifications for a user to be alerted. According to this disclosure, the system and method may be configurable such that the template screens on a mobile device (31) application (34) will include at least a means to enter at least one time and date for a notification, as well as whether the notification is to repeat or not and at what frequency the notification is to repeat, which can be selected on the template screen, via selection of a check box, radio button, or the like as well as a similar selection to stop the selected notification. In some embodiments, the system and method will additionally be configured such that multiple times and dates may be selected for multiple, independent notifications with at least one means to turn off, reset, or deselect the notification once the preselected notification time and day are reached. In these alternative embodiments, the system and method may be configured such that each independent notification has the selection of times, dates, repeating or one time notifications, frequency of notification repeating, as well as deselecting, resetting, or turning off a notification or even all notifications. Other embodiments may include different and distinct notifications for each independent notification, including different lighting patterns, different colored lights, different text notifications, different picture notifications, or even combinations and/or portions thereof.

When the preselected time and day are met, the system sends a notification to the user through either the pet wearable device, the user mobile device, or both. In some instances of multiple notifications, or repeating notifications, one or more numerical quantities representing associated notification condition thresholds may be entered on the template screen. The system may be configured so that, when a notification occurs, the system automatically sends notification through LED (1) lights to notify the user.

The notification may comprise LED (1) lights, an alphanumeric message sent to a portable wireless communication device, such as a pager, PDA, wireless communication badge, wireless phone handset, or any other portable wireless device having text messaging capability. The notifications may also comprise pictures, icons, images or even portions and/or combinations of the foregoing. Alternatively or additionally, the notification may be a system-generated audio message to a portable wireless communication device, such as those mentioned previously.

A network (35) interface through the user mobile computing device (31) may be configured to communicate data to an external storage database (36) or other computer for programming use, memory storage, or both. Data may also include pet medical history. In come embodiments, the network (35) interface can be used to transmit the notifications to the pet wearable device (30) directly, or the notifications may be stored on directly on the pet wearable device memory. (23) The training data, or programable notification schedule, may therefore be entered wirelessly into the pet wearable device (30) through either an external computer (not shown), the user mobile device (30) through the mobile device application (34), or even through a wired connection between the computer and the pet wearable device (30) or between the user mobile computing device (31) and the pet wearable device. (30)

In other words, in the preferred embodiment, the light emitted from the pet wearable device LED (1) helps the pet owner to see the reminder signal for the predetermined event (such as the administration of medication). Additionally, in other embodiments, the system and method are configured such that the pet owner can program the application on the mobile device to set times to be reminded by an audible alarm, text, images, pictures, icons, portions of the foregoing, or a combination thereof, to as notifications to the user, such as a notification to administer medication to the pet.

When the preselected, programmed notification day and time is reached, the notification is activated. In the preferred embodiment, the notification consists of the LED (1) light being activated on the pet wearable device.

The on/off switch (3) on the pet wearable device may comprise a button, switch, slide, rocker, or similar actuating mechanism and, in some embodiments, is used to select the cessation of a notification, and, in other embodiments, more than one notification. The said cessation of the notification, as referred to in this paragraph, should be understood that the notification may be deselected, turned off, or reset for a subsequent notification in the case of multiple notifications or repeating notifications. The method and system are configured so that the selection of the use of the mobile device to turn off the notification, the pet wearable device to turn off the notification, of both the on/off switch on the pet wearable device and selection on the user mobile device application to turn off the notification. The preferred embodiment requires the user to turn off the notification on the user's mobile device application as well as to use the on/off switch on the pet wearable device to turn off the notification. As explained elsewhere in the document, this two-factor requirement for turning off the notification is intended to help compliance with the desired action coinciding with the preselected notification. In embodiments including screens to display text, pictorials, images, icons, or a combination of these for notifications, the use of the pet wearable switch, mobile device application, or both the pet wearable switch and mobile device application likewise deselect, reset or turn off the notification. In other embodiments, different notifications of lights, texts, pictorials, images, icons or a combination may be configured and selected for different notifications. In other embodiments, default templates may be configured for common time intervals and notification regimes, such as twice a day for insulin administration or even every two hours for blood glucose sampling for a diabetic pet.

In accordance with the preferred embodiment of the system and method, as explained herein, the notification parameters are entered into the mobile communication device application and thereafter, the system and method notifications and notification cessations operate in accordance with the application software to notify within the notification parameters stored in memory. In other embodiments, as also mentioned herein, one or more computer devices, such as PC's or servers or any other devices capable of executing software, are included as part of the method and system to program the notifications, store the notification data, and turn off/reset/deselect notifications and connect to the network.

As an example of a use of the preferred embodiment, a pet owner receives news that a pet has a condition that requires medication. As an illustration, a pet dog may be diagnosed with diabetes. As a result, the veterinarian, besides adjusting the dog's diet, will likely prescribe a dosage of insulin for the dog. This dosage may well be diagnosed after the pet owner takes blood tests of the dog on a regular basis throughout the day to monitor the dog's sugar levels. The present invention could be used as the owner opens their mobile phone application and programs the LED lights to signal and remind the owner to take a blood sample every two hours. If busy and the means to shut off the reminder signal is on the app, the owner could possibly turn off the signal on the application and inadvertently become distracted and forget to take the blood sample, thereby potentially aborting the test. Further, once the insulin dosage is determined with accurate glucose levels of the dog, the application could be set to twice a day after meals for insulin injection into the dog to keep the dog healthy. The importance of following medication administration at the predetermined dosages and intervals is well understood; in this example, the reminders help ensure the diabetic pet is not given too much insulin, (causing hypoglycemia and potentially convulsions, and death) or given too little insulin (causing hyperglycemia, diabetic coma, and death).

The pet wearable device comprises a method of programing an event schedule into a first apparatus, which corresponds to a user's mobile device. The event schedule includes at least one repeating time cycle for a required action. This schedule is transmitted using a plurality of signals from this first apparatus, wherein at least one signal includes the event schedule from the first apparatus to a second apparatus. The second apparatus may correspond to the pet wearable device in the preferred embodiment. Both the first and second apparatus must have a power supply and be turned on. The first apparatus is designed to detect the second apparatus for the signal transmissions. As is common in wireless communications, a security protocol, such as encryption, is employed in the transmissions and is activated in a transmission and receiving protocol in the first and second apparatuses. An ensuing match-based connection is established between the first apparatus and the second apparatus and the event schedule from the first apparatus is received by the second apparatus. The second apparatus stores this event schedule, called training data, because the data comprises the event schedule data and trains the second device. The first device also retains the event schedule from the initial programming. The storage may be use one or more machine-learning techniques to train a machine-learned model based on the training data, wherein the machine learned model is configured to generate an output associated with at least one of the first apparatus and the second apparatus. Once the event scheduled is learned, and the predetermined time for the event is reached, a notification is sent to alert the user that the predetermined time for the event has been reached. The notification alert is then turned off by the user so that the notification does not continue once the user reminder has been achieved.

Turning off the event schedule notification can turn off the event schedule notification of the first apparatus, turn off the event schedule notification of the second apparatus, or turn off the event schedule notification of the first and second apparatus.

The notification device is a lighting mechanism configured to emit one or more light indications from the second apparatus. The communication between the first device and the second device can include, but is not limited to, UWB, BLE, Bluetooth, Wifi, or a combination of UWB and BLE.

In another embodiment, the second apparatus can be programmed an event schedule into the second apparatus directly with an event schedule that includes at least one repeating time cycle for an event action. The first apparatus may further comprise a screen to display messages. The second apparatus may also further comprise a screen to display messages. The first apparatus may also receive the event schedule notifications.

The second apparatus may attach to a pet collar. The attachment may include the use of strap, screw, buckle, rivet, or hook and loop fastener.

In another embodiment, the pet wearable device may be at least partially flexible, comprising at least one of the group consisting of: flexible PCB, flexible battery array, flexible housing, LED, and flexible solder.

The present invention is also a system comprising a pet reminder collar device input system adapted to receive event notification schedule input (for example, medication administration regime input) and execute computer instructions to receive and store the event notification schedule input (again as an example, medication administration regime). The collar device has a housing that includes a processor communicatively coupled to at least one mobile communication device; at least one lighting mechanism configured to emit one or more light indications notifications; a battery; a switch to turn off the event notification schedule (such as a medication administration) notification; and a connection means to attach said collar device to a pet collar. The mobile communication device is adapted to accept a plurality of programmed computer instructions comprising an event notification schedule (such as a medication administration regime) input, store said event notification schedule (such as medication administration regime), detect, communicatively couple, and transmit said computer instructions comprising the event notification schedule (such as medication administration regime) to said pet reminder collar device; execute a notification communication according to event notification schedule (such as medication administration regime); and, stop the notification communication when the mobile communication device user activates the switch to turn off the event notification schedule (such as medication administration regime) notification.

The battery can be rechargeable, and the device contains at least one charging port to recharge the battery. In addition, the system can have more than one event notification schedule, which includes a plurality of medication administration regimes.

The input system can be configured to receive data input in the form of notifications such as medication administration regime, text, and pictures. Additionally, the pet reminder collar device may also have a pet two-tier compliance factor reminder collar device input system that requires the notifications to be turned off on said mobile device as well as said pet collar device housing.

The method of the event notification schedule, like the medication administration regime method, can also include a security mode transmission and receiving protocol on the first and second apparatuses as well as the event notification schedule comprising a plurality of event notifications.

In alternative embodiments, multiple lights, light patterns, or other distinguishing signals can indicate different reminders on the collar light. In further embodiments, the pet owner can program the application on the smart device for multiple alarms. In yet another embodiment, the pet owner can program the app to choose one or more of the following: audible alarm on the app and smart device, audible alarm on the collar light, different light patterns of the collar light, different color light patterns on the collar light, different messages in the app, and different days, times, and schedules on the app. In other embodiments, the app provides a means to contact the veterinarian, request refills on medication, and automatically requests refills on medication. In other embodiments, the pet collar light contains at least one repository for pet medication and contains buttons that allow for the programming of the reminder messages, times, and days.

While the invention has been described in connection with a preferred embodiment, it is not intended to limit the scope of the invention to the form set forth, but on the contrary, it is intended to cover such alternatives, modifications, and equivalents as may be included within the spirit and scope of the invention as defined by the appended claims.

1. A method, comprising:
programing an event schedule into a first apparatus,
wherein said event schedule includes at least one repeating time cycle for a required action;
transmitting a plurality of signals from the first apparatus,
wherein at least one signal includes the event schedule from the first apparatus to a second apparatus;
detecting a second apparatus;
activating a security mode transmission and receiving protocol on the first and second apparatuses;
establishing a match-based connection between the first apparatus and the second apparatus;
receiving the event schedule from the first apparatus by the second apparatus;
storing training data,
wherein said training data comprises the event schedule data;
utilizing one or more machine-learning techniques to train a machine-learned model based on the training data,
wherein the machine-learned model is configured to generate an output associated with at least one of the first apparatus and the second apparatus;
sending a notification when an event action is indicated by the event schedule; and,
turning off the event schedule notification.

2. The method of claim 1, turning off the event schedule notification on one of the group consisting of the following: turning off the event schedule notification of the first apparatus, turning off the event schedule notification of the second apparatus, or turning off the event schedule notification of the first and second apparatus.

3. The method of claim 1, wherein the second apparatus is a pet wearable device containing a processor and a notification device.

4. The method of claim 3, wherein the notification device is a lighting mechanism configured to emit one or more light indications from the second apparatus.

5. The method of claim 1, wherein said signal is selected from the group consisting of: UWB, BLE, Bluetooth, Wifi, or a combination of UWB and BLE.

6. The method of claim 1, programming an event schedule into the second apparatus, said event schedule includes at least one repeating time cycle for an event action.

7. The method of claim 1, wherein the first apparatus further comprises a screen to display messages.

8. The method of claim 7, wherein the second apparatus further comprises a screen to display messages.

9. The method of claim 1, wherein the first apparatus also receives the event schedule notifications.

10. The method of claim 3, wherein the pet wearable device attaches to a pet collar.

11. The method of claim 10, wherein the pet wearable device attaches as selected from the group consisting of at least one of the following: strap, screw, buckle, rivet, or hook and loop fastener.

12. The method of claim 3, wherein said pet wearable device is comprised of at least one of the group consisting of: flexible PCB, flexible battery array, flexible housing, LED, and flexible solder.

13. A system comprising:
a pet reminder collar device input system adapted to receive medication administration regime input and execute computer instructions to:
receive and store said medication administration regime;
said collar device having a housing configured to include:
a processor communicatively coupled to at least one mobile communication device;
at least one lighting mechanism configured to emit one or more light indications notifications;
a battery;
a switch to turn off the medication administration notification; and,
a connection means to attach said collar device to a pet collar;
said mobile communication device adapted to accept a plurality of programmed computer instructions comprising medication administration regime input, store said medication administration regime, detect, communicatively couple, and transmit said computer instructions comprising medication administration regime to said pet reminder collar device;
execute a notification communication according to medication administration regime; and,
stop the notification communication when the mobile communication device user activates the switch to turn off the medication administration notification.

14. The system of claim 13, wherein said battery is rechargeable.

15. The system of claim 14, wherein said collar device contains at least one charging port.

16. The system of claim 13, further comprising a plurality of medication administration regimes.

17. The system of claim 13, further comprising said input system configured for one or more of the group consisting of medication administration regime, text, and pictures.

18. The system of claim 13, wherein the pet reminder collar device further comprises a pet two-tier compliance factor reminder collar device input system requiring the notifications to be turned off on said mobile device and said pet collar device housing.

19. A method, comprising:
programing an event notification schedule into a first apparatus,
wherein said event notification schedule includes at least one repeating time cycle for event notification;
transmitting a plurality of signals from the first apparatus,
wherein at least one signal includes the event notification schedule from the first apparatus to a second apparatus;
detecting a second apparatus;
establishing a match-based connection between the first apparatus and the second apparatus;
receiving the event notification schedule from the first apparatus by the second apparatus;
storing training data,
wherein said training data comprises the event notification schedule data;
utilizing one or more machine-learning techniques to train a machine-learned model based on the training data,
wherein the machine learned model is configured to generate an output associated with at least one of the first apparatus and the second apparatus;
sending a notification when an event notification schedule is indicated by the event notification schedule; and,
turning off the event notification schedule notification of the first and second apparatus.

20. The method of claim 20 further comprising activating a security mode transmission and receiving protocol on the first and second apparatuses.

21. The method of claim 20 wherein said event notification schedule comprises a plurality of event notifications.
